SEQUENCE LISTING (11 sequences)

SEQ ID NO: 1 - protein, 77 aa, Homo sapiens
RSVRAQRHTAMPKTQKYQPPSTNKNTKSQRRKGWPKTHPGGQQKQGTEASLQIRGKKKEQRREIGSRNAECRGKKGK

SEQ ID NO: 2 - protein, 74 aa, Oncorhynchus mykiss
RSVRAQRHTDMPRTPKVSTAVQNVDRGTERRTAGHPDKTKTKKKPLSGHSHPSCKEVHQKNSSRGNTGGRNTRM

SEQ ID NO: 3 - protein, 62 aa, Oncorhynchus mykiss
RSVRAQRHTDMPRTPKVSTAVQSVDRGTERRTAGHPDKTKPKKEVHQKNSSRGNTGGRNTRM

SEQ ID NO: 4 - protein, 47 aa, Oncorhynchus mykiss
RSVRAQRHTDMPRTPKKPLSGHSHPSCKEVHQKNSSRGNTGGRNTRM

SEQ ID NO: 5 - protein, 35 aa, Oncorhynchus mykiss
RSVRAQRHTDMPRTPKEVHQKNSSRGNTGGRNYRM

SEQ ID NO: 6 - DNA, 18 bp, Artificial
  Synthetic Primer
cttgtggccgtttacgtc

SEQ ID NO: 7 - DNA, 18 bp, Artificial
  Synthetic Primer
gcacagcacccagacaag

SEQ ID NO: 8 - protein, 74 aa, Rattus norvegicus
  MISC_FEATURE (1)..(74): rat Ea4 peptide
RSVRAQRHTDMPRTPKVSTAVQNVDRGTERRTAQHPDKTKTKKKPLSGHSHPSCKEVHQKNSSRGNTGGRNYRM

SEQ ID NO: 9 - protein, 60 aa, Rattus norvegicus
  MISC_FEATURE (1)..(60): rat Ea3 peptide
RSVRAQRHTDMPRTPKVSTAVQSVDRGTERRTAQHPDKPKKEVHQKNSSRGNTGGRNYRM

SEQ ID NO: 10 - protein, 47 aa, Rattus norvegicus
  MISC_FEATURE (1)..(47): rat Ea2 peptide
RSVRAQRHTDMPRTPKKPLSGHSHPSCKEVHQKNSSRGNTGGRNYRM

SEQ ID NO: 11 - protein, 35 aa, Rattus norvegicus; identical to SEQ ID NO: 5
  MISC_FEATURE (1)..(35): rat Ea1 peptide

(19) United States
(12) Patent Application Publication
(10) Pub. No.: US 2009/0318355 A1
(43) Pub. Date: Dec. 24, 2009

(54) COMPOSITIONS AND METHODS FOR PROMOTING TISSUE REPAIR AND WOUND HEALING

(76) Inventors: Thomas T. Chen, Storrs, CT (US); Maria J.M. Chen, Storrs, CT (US)

Correspondence Address: MCCARTER & ENGLISH, LLP HARTFORD;CITYPLACE I, 185 ASYLUM STREET, HARTFORD, CT, 06103, US

(21) Appl. No.: 12/465,065

(22) Filed: May 13, 2009

Related U.S. Application Data
(63) Continuation-in-part of application No. 11/354,484, filed on Feb. 15, 2006.
Application No. 11/354,484 is a continuation-in-part of application No. 11/799,623, filed on May 2, 2007.
(60) Provisional application No. 61/052,779, filed on May 13, 2008.
(60) Provisional application No. 61/052,781, filed on May 13, 2008.

(51) Int. Cl. A61K 38/18; A61P 17/02

(52) U.S. Cl. 514/12

(57) ABSTRACT

A method is described for using the Ea4-peptide of pro-IGF-I or human Eb-peptide of pro-IGF-I for enhancing the proliferation of fibroblasts and closure of wound. The peptide species can be homologous of trout Ea4-peptide, human Eb-peptide of pro-IGF-I or a fusion protein comprising the Ea4- or Eb-peptide of pro-IGF-I. It can be administered any wound in a pharmaceutically acceptable composition alone or in combination with other compounds.

### CROSS-REFERENCE TO RELATED APPLICATIONS

Under 35 U.S.C. § 119(e) this application claims the benefit to U.S. Provisional Patent Applications 61/052,779 and 61/052,781 filed: May 13, 2008; and is a Continuation-in-Part of U.S. patent application Ser. No. 11/354,484 filed Feb. 15, 2006, entitled: Compositions and methods for inducing apoptosis in tumor cells; and U.S. patent application Ser. No. 11/799,623 filed May 2, 2007, entitled: Anti-tumor activity of Ea-4-peptide of pro-IGF-1; the disclosures of which are hereby incorporated by reference in their entirety.

### SEQUENCE LISTING

The present application hereby incorporates by reference, in its entirety, the Sequence Listing submitted herewith. An electronic version of the Sequence Listing is being filed herewith, file name: 97511_{—}00011_ST25.txt, size: 7 KB, created: May 13, 2009 using PatentIn 3.4 software on Windows XP; the contents of which are identical to the written version of the Sequence Listing.

### FIELD OF THE INVENTION

The present invention relates generally to therapeutic uses of IGF-I peptides. In particular, the present invention relates to use of IGF-I, E-domain peptides for promoting wound healing and tissue repair.

### BACKGROUND

In response to external damage and internal degeneration, the cells of the body must repair the tissues surrounding the each cell in order to maintain their function and the health of the organism. Defects in the ability to repair tissues has been linked to many diseases and pathological conditions, for example, neurodegenerative diseases (e.g., Parkinson's Disease), heart attacks, heart failure, muscular dystrophy, bed sores, diabetic ulcers, oxidative damage, burns, lacerations, abrasions, and tissue damage such as sinusitis that occurs as side effect from the administration of chemotherapeutic agents.

The mature form of IGF-I is a basic protein of 7.5-kDa. The pre-pro-peptides of the IGF-I consist of an amino-terminal signal peptide, followed by the mature peptide with B, C, A and D domains, and a carboxyl-terminal E domain (See FIG. 1A for a schematic representation). The signal peptide at the amino-terminal end and the E-domain peptide at the carboxy-terminal end of the pre-pro-peptide are proteolytically cleaved from the peptide to result in the mature, biochemically active species. Tian et al. (1999) have reported that recombinant rainbow trout Ea-2-, Ea-3- and Ea-4-peptides possess mitogenic activity in several non-transformed cell lines, including NIH 3T3 cells and caprine mammary epithelium cells (CMEC) (Panschenko et al., 1997). Trout Ea-2- and Ea-4-peptide contains a signal motif for peptidyl C-terminal amidation (Shamblott and Chen, 1993; Barr, 1991), and a bipartite consensus nuclear localization sequence is also present in Ea-4-peptide (Shamblott and Chen, 1993; Dingwall and Laskey, 1991). The present inventors have now discovered that the E-peptides possess novel biological activities including the promotion of wound healing and tissue repair.

Accordingly, the present invention is particularly useful because of the ongoing need for novel compositions and methods to improve the regenerative capacity of various tissues for the treatment of conditions related to acute and chronic cellular and tissue damage.

### SUMMARY

Described herein are compositions and methods for promoting repair and/or healing of tissue and/or cellular damage (i.e., wounds) in an animal. The present invention is based on the surprising and unexpected discovery that insulin-like growth factor-1 (IGF-1) E-domain peptides (or “E-peptides”) promote the proliferation and migration of fibroblast cells, which form the stroma or supporting matrix for most organs, for example, the skin. Therefore, in one aspect, the invention relates to compositions and methods for promoting fibroblast proliferation comprising treating a fibroblast with a composition comprising an effective amount of an E-domain peptide together with at least one of a pharmaceutically acceptable carrier, excipient or adjuvant, wherein the composition effectuates fibroblast proliferation.

In another aspect, the invention relates to compositions and methods for promoting repair or healing of tissue and/or cellular damage (i.e., wounds) in an animal comprising administering a composition comprising an effective amount of an E-domain peptide together with at least one of a pharmaceutically acceptable carrier, excipient or adjuvant, wherein the composition effectuates repair of the wound.

In another aspect, the invention relates to nucleic acid constructs (i.e., plasmids, vectors, expression constructs, and the like) comprising a nucleic acid encoding for an E-domain polypeptide or fragment thereof, operably linked with at least one DNA regulatory element, for example, a transcription, and/or replication regulatory element.

In another aspect, the composition comprises at least one E-peptide having the amino acid sequence with at least about 30%, 40%, 50%, 60%, 70%, 80%, or 90% sequence homology with an hEb-domain peptide having the amino acid sequence of SEQ ID NO: 1.

Other aspects and advantages of the present invention will be readily apparent in view of the following Detailed Description, accompanying Drawings, and claims.

### BRIEF DESCRIPTION OF THE DRAWINGS

The accompanying drawings, which are incorporated into and form a part of the specification, illustrate several aspects of the present invention and, together with the description, serve to explain the principles of the invention. The drawings are only for the purpose of illustrating embodiments of the invention and are not to be construed as limiting the invention.

FIG. 1 (A) is a schematic representation of the subforms of mammalian and rainbow trout pro-IGF-1 pro-peptides. B, C, A, D, and E indicate different domains of the IGF-1 peptides. (B) shows the amino sequence alignment of hEb (SEQ ID NO: 1), rtEa-4 (SEQ ID NO:2), rtEa-3 (SEQ ID NO:3), rtEa-2 (SEQ ID NO: 4), and rtEa-1 (SEQ ID NO: 5).

FIG. 2 is a schematic representation of the gene constructs used in the transfection of target cells. IGF-1-sp: signal peptide of hlGF-I; Ea-4 cDNA: cDNA of rtEa-4-peptide; hEb cDNA: cDNA of the human Eb peptide; EGFP: coding region of the enhanced green fluorescence protein gene; IRES: internal ribosome entry site.

FIG. 3 Effect of rtEa4-peptide on scratch wound closure of human fibroblast, CCD-1112SK, cells (ATCC® Number: CRL-2429). Human fibroblast cells were grown to monolayer in 6-well plates, scratches were made with an one ml pipette tip, and various amounts of rtEa4-peptide were supplemented into the growth medium. After 24 hours, the medium was removed and the cells were stained by Giemsa stain and observed under a microscope. A dose-dependent effect on scratch wound closure by rtEa4-peptide was observed.

FIG. 4 Effect of shEb-peptide on scratch wound closure of human fibroblast, CCD-1112SK, cells. Human fibroblast cells were grown to monolayer in 6-well plates, scratches were made with an one ml pipette tip, and various amounts of shEb-peptide were supplemented into the growth medium. After 36 hours, the medium was removed and the cells were stained by Giemsa stain and observed under a microscope. A dose-dependent effect on scratch wound closure by rtEa4-peptide was observed.

FIG. 5 Effect of E-peptides on levels of c-jun mRNA in human fibroblast cells. Human fibroblast, CCD-1112SK, cells were grown to monolayer in 6-well plates, scratches were made with an one ml pipette tip, and various amounts of E-peptides were supplemented into the growth medium. After 6 hours, the cells were harvested and total RNA extracted by the guanidinium thiocyanate-phenol-chloroform method. Level of c-jun and beta-actin mRNAs were determined by real-time quantitative PCR (RTQ-PCR) method and data expressed as: Relative mRNA level=2^{-[S·CT-C·CT]}. Each data point was the average of at least 6 determinations. The levels of c-jun mRNA in the scratched wound of CCD-1112SK cells were significantly induced by E-peptides.

FIG. 6 Effect of signal transduction pathway inhibitors on levels of rtEa4-peptide induced c-jun mRNA in human fibroblast cells. Human fibroblast, CCD-1112SK, cells were grown to monolayer in 6-well plates, scratches were made with an one ml pipette tip, treated with various concentrations of signal transduction pathway inhibitors for 1 hours, and followed by 6 hour with rtEa4-peptide (about 10 μg/ml). Cells were harvested, and total RNA extracted with the guanidinium thiocyanate-phenol-chloroform method. Levels of c-jun and beta-actin mRNAs were determined by RTQ-PCR assay. Relative mRNA level=2^{-[S·CT-C·CT]}. Each data point was the average of at least 6 determinations. UO126 and PD098059, inhibitors of Med1/2; SP600125, inhibitor of JNK; SB203580, inhibitor of P38 MAPK. The data showed that the rtEa4-peptide induced c-jun mRNA was suppressed by inhibitors of signal transduction pathways.

FIG. 7 Effect of signal transduction pathway inhibitors on rtEa4-peptide induced scratch wound closure in human fibroblast, CCD-1112SK, cells. Human fibroblast cells were grown to monolayer in 6-well plates, scratches were made with an one ml pipette tip, treated with signal transduction pathway inhibitors for 1 hour, and 10 μg/ml of rtEa4-peptide were supplemented into the growth medium. After 24 hours incubation, medium was removed and cells are stained with Giemsa stain and observed under a microscope. (a) CCD-1112SK cells in growth medium; (b) CCD-1112SK cells with rtEa4-peptide alone; (c) CCD-1112SK cells with rtEa4-peptide and PD098059; (d) CCD-1112SK cells with rtEa4-peptide and SP6001254; (e) CCD-1112SK cells with rtEa4-peptide and UO126; (f) CCD-1112SK cells with rtEa4-peptide and SB203580. Inhibitors of signal transduction pathways abolished rtEa4-peptide induced scratch wound closure in CCD-1112SK cells.

FIG. 8 Effect of rtEa4-peptide on suppression of PTEN mRNA level in human fibroblast, CCD-1112SK, cells. Human fibroblast cells were grown to monolayer in 6-well plates, scratches were made with an one ml pipette tip, and treated with 10 μg/ml of rtEa4-peptide. After 6 hours incubation, cells were harvested and total RNA extracted by the guanidinium thiocyanate-phenol-chloroform method. Levels of PTEN and beta-actin mRNAs were determined by RTQ-PCR assay. Relative mRNA level=2^{-[S·CT-C·CT]}. Each data point was the average of at least 6 determinations. The level of PTEN mRNA in the scratched wound of CCD-1112SK cells was significantly suppressed by rtEa4-peptide.

### DETAILED DESCRIPTION

Described herein are compositions and methods for promoting repair and/or healing of tissue and/or cellular damage (i.e., wounds) in an animal. The present invention is based on the surprising and unexpected discovery that Insulin-like Growth Factor-1 (IGF-1) E-domain peptides (or E-peptides) promote the growth of fibroblast cells, which form the stroma or supporting matrix for most organs, including the skin. The growth promoting activity is demonstrated in response to physical perturbation of fibroblast cells. This biological activity, which could not have been predicted, a priori, indicates that E-peptides are useful for promoting the repair or healing of tissue and/or cellular damage.

The following U.S. patent applications and U.S. patents discuss subject matter related to the present invention and are incorporated herein by reference: U.S. patent application Ser. No. 11/354,484 filed Feb. 15, 2006; U.S. patent application Ser. No. 11/799,623 filed May 2, 2007; and U.S. Pat. Nos. 6,358,916; 6,610,302; 7,118,752; and 7,250,169.

Insulin-like growth factors (IGF's) are mitogenic peptides that regulate embryonic development, post-natal growth and cellular differentiation in vertebrates. The functions of mature IGF peptides have been extensively studied in various in vitro and in vivo systems. IGF's, including IGF-I and IGF-II, are among the members of a family of structurally and evolutionarily related peptides that also include insulin and relaxins. Like many hormones, IGF's are initially translated as pre-pro-peptides that undergo post-translational processing to result in the mature peptides.

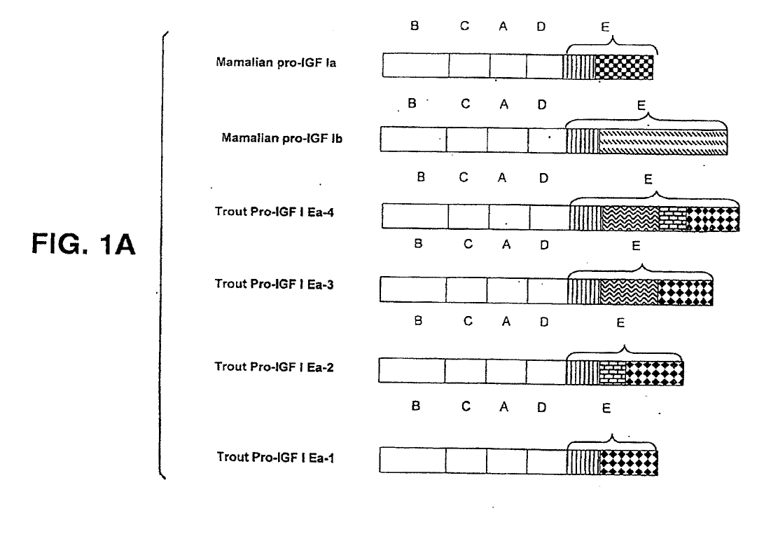

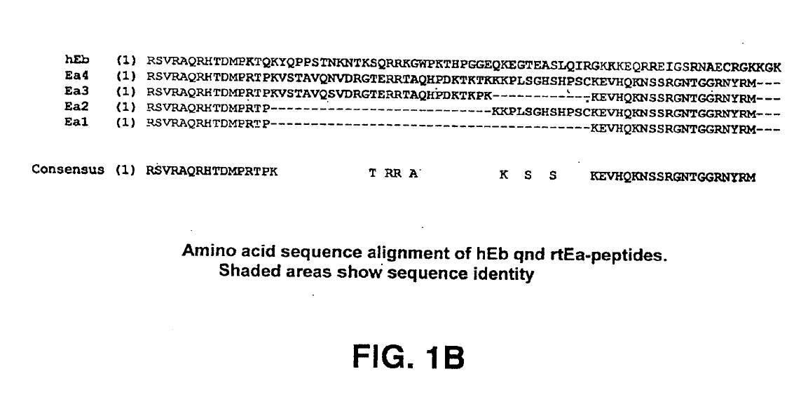

The mature form of IGF-I is a basic protein of 7.5 kDa. The pre-pro-peptides of the IGF-I consist of an amino-terminal signal peptide, followed by the mature peptide with B, C, A and D domains, and a carboxyl-terminal E domain (See FIG. 1A). The signal peptide at the amino-terminal end and the E-domain peptide at the carboxy-terminal end of the pre-pro-peptide are proteolytically cleaved from the peptide to result in the mature, biochemically active species.

To date, multiple forms of pro-IGF-I have been identified in species from fish to mammals (Shamblott, Chen, Mol Mar Biol Biotechnol. 2: 351-61, 1993; Rotwein, Proc. Natl. Acad. Sci. USA, 83:77-81, 1986). See, for example, accession numbers (Uniprot or NCBI): P16501 (Xenopus laevis), P05017 (Mus musculus), Q95222 (Oryctolagus cuniculus), P08025 (Rattus norvegicus), Q90325 (Cyprinus carpio), CAA40092 (Homo sapiens), NP_{—}001071296 (Bos taurus), P10763 (Ovis aries); P16545 (Sus scrofa); Q02815 (Oncorhynchus mykiss); P17085 (Oncorhynchus kisutch); P18254 (Gallus gallus); P51458 (Equus caballus); NP_{—}571900 (Danio rerio); and P33712 (Canis familiaris); which are incorporated herein by reference.

In humans, three alternative spliced isoforms of pro-IGF-I (pro-IGF-1-a pro-IGF-I-b and pro-IGF-I-c) have been reported (Rotwein, Proc. Natl. Acad. Sci. USA, 1986; Rotwein, et al., J. Biol. Chem., 261: 4828-32, 1986; Chew, et al., Endocrinology, 136: 1939-44, 1995). These three pro-IGF-I isoforms differ only in the carboxyl-terminal E-domain regions that are normally removed in vivo from the mature IGF-I. The E-domains of pro-IGF-I-a, pro-IGF-I-b and pro-IGF-I-c contain 35, 77 and 40 amino acid residues, respectively. The first 15 amino acid residues at the N-terminus of E-domains (referred to as the common region) share identical sequences. The amino acid sequences following the common region vary between the three isoforms of human pro-IGF-I (see FIG. 1B).

Similar diversity of pro-IGF-I E-domains is also found in rainbow trout (Oncorhynchus mykis), where four different isoforms have been identified, designated for consistent reference herein as pro-IGF-I Ea-1, Ea-2, Ea-3 and Ea-4 (Shamblott, Chen, Mol. Mar. Biol. Biotech., 1993). Nucleotide sequence comparison of the four size forms of rainbow trout IGF-I mRNAs is consistent with the above observations concerning the Ea peptides in that the size differences among these mRNA species are due to insertions or deletions in the E domain regions of the molecules (See FIGS. 1A and 1B). The predicted amino acid residues of the common region of the four Ea peptides share identical sequences among themselves, as well as with pro-IGF-I E-peptides of human, mouse, and rat species (See FIG. 1B). The presence of the C-terminal 20 amino acid residues, sharing 70% identity with their human counterparts, identifies them as a-type E-peptides. The Ea-1 peptide of the rainbow trout (rt) pro-IGF-I (SEQ ID NO: 5) is a polypeptide of 35 amino acid residues, comprising the first 15 and the last 20 amino acid residues. Ea-2 (SEQ ID NO: 4) and Ea-3 (SEQ ID NO: 3) peptides differ from Ea-I by virtue of either a 12- or 27-amino acid residue insertion between the first and last segments of the Ea-1-peptide sequence, respectively (see FIG. 1B). The Ea-4 peptide (SEQ ID NO: 2) contains both insertions. The predicted numbers of amino acid residues in each E-peptide are, thus, 35 (SEQ ID NO: 5), 47 (SEQ ID NO: 4), 62 (SEQ ID NO: 3) and 74 (SEQ ID NO: 2), respectively. There has not been any report on the presence of b-type IGF-I mRNA in rainbow trout (Shamblott and Chen, 1993).

FIG. 1B shows the amino acid sequences of the human Eb peptide (hEb) (SEQ ID NO:1) and the trout Ea peptides. Despite not having complete homology at the primary level, preliminary studies (unpublished data of this laboratory) indicate that hEb and trout Ea-4 peptide have very similar tertiary structures, particularly in the amino-terminal region containing the common sequences, and can compete effectively for binding to cell receptors specific to E-domain peptides.

Despite the presence of multiple E-domain variants, assigning biological function to the IGF E-domains has been elusive. Proteolytic processing of the pro-IGF's, resulting in the cleavage of E-domains from IGF's, is believed to be similar to the cleavage of the C-peptide of proinsulin (Foyt, et al., Insulin-Like Growth Factors: Molecular and Cellular Aspects, pp 1-16. Boca Raton: CRC press, 1991). In the past, it was generally accepted that E-domains, like the C-peptide of pro-insulin, possess little or no biological activity other than their potential roles in the biosynthesis of mature IGF. The C-peptide of pro-insulin is believed to have an essential function in the biosynthesis of insulin in linking the A and B chains in a manner that allows correct folding and inter-chain disulfide bond formation. In spite of the earlier reports indicating certain physiological effects of the insulin C-peptide (Johansson, et al., Diabetologia, 35: 121-28, 1992; Johansson, et al., Diabetologia, 35: 1151-58, 1992; Johansson, et al., J. Clin. Endo. Metab., 77: 976-81, 1993), it has not been widely accepted until recently. The C-peptide has now been shown to have many beneficial effects on various abnormalities in diabetic animal models and patients (Ido, et al., Science, 277: 563-66, 1997; Forst, et al., J. Clin. Invest. 101: 2036-41, 1998; Sjoquist, et al., Kidney Int., 54: 758-64, 1998). Moreover, recent studies further demonstrated specific binding of C-peptide to cell surfaces in a manner that suggests the presence of G-protein-coupled membrane receptors (Rigler, et al., Proc. Natl. Acad. Sci. USA, 96: 13318-23, 1999). It is now thought that C-peptide may thereby stimulate specific intracellular signal transduction leading to the biological activities of C-peptide (Wahren, et al., Am. J. Physiol. Endo. Metab. 278: E759-68, 2000; Kitamura, et al., Biochem J., 355: 123-29, 2001).

Recombinant Ea-2, Ea-3 and Ea-4 peptides of rainbow trout pro-IGF-I possess mitogenic activity in cultured BALB/3T3 fibroblast (Tian, et al., Endocrinology, 140: 3387-90, 1999). In addition to mitogenic activity, trout pro-IGF-I Ea-2 and Ea-4 peptides possess activities including induction of morphological change, enhancement of cell attachment, restoration of anchorage-dependent cell division behavior, and reduction of the invasiveness of aggressive cancer cells. Since similar morphological change has also been induced in a hepatoma cell line of Peoceliposis lucida (desert guppy) by treatment with the trout Ea-4 peptide, this observation rules out the possibility that the effects of trout pro-IGF-I Ea-4-peptide on human cancer cells are the consequence of artifact.

A fibroblast is a type of cell that synthesizes and maintains the extracellular matrix of many animal tissues. Fibroblasts provide a structural framework (stroma) for many tissues, and play a critical role in wound healing. They are the most common cells of connective tissue in animals. While epithelial cells form the lining of body structures, it is fibroblasts and related connective tissues which sculpt the “bulk” of an organism.

The main function of fibroblasts is to maintain the structural integrity of connective tissues by continuously secreting precursors of the extracellular matrix. Fibroblasts secrete the precursors of all the components of the extracellular matrix, primarily the ground substance and a variety of fibres. The composition of the extracellular matrix determines the physical properties of connective tissues. Fibroblasts make collagens, glycosaminoglycans, reticular and elastic fibers, and glycoproteins found in the extracellular matrix. Growing individuals' fibroblasts are dividing and synthesizing ground substance. Tissue damage stimulates fibrocytes and induces the mitosis of fibroblasts. Active fibroblasts can be recognized by their abundant rough endoplasmic reticulum. Inactive fibroblasts, which are also called fibrocytes, are smaller and spindle shaped. They have a reduced rough endoplasmic reticulum. Although disjointed and scattered when they have to cover a large space, fibroblasts when crowded often locally align in parallel clusters.

The present invention confirms that the rtEa-4-peptide and hEb-peptide have novel biological activities. These activities include induction of morphological change, migration and proliferation of fibroblast cells, and therefore, the E-peptides of the invention and therapeutic compositions comprising the same are useful for promoting repair of damaged tissue, for example, in an animal, and/or repair of cellular damage. The present discovery is surprising and unexpected because heretofore, it has not been demonstrated that human Eb-peptide or rtEa4-peptide were capable of inducing the proliferation/migration of fibroblasts, in particular, human fibroblasts. Moreover, due to the unpredictability inherent in the art, the present results could not have been predicted.

The present invention provides a method and compositions comprising IGF-I E-domain peptides with utility for promoting the proliferation of fibroblast. In particular, the present invention describes methods and compositions for promoting cellular and/or tissue repair, e.g., wound healing, in vitro and in vivo. A method is described for using the rtEa4-peptide of pro-IGF-I or human Eb-peptide of pro-IGF-I for enhancing the proliferation of fibroblasts and closure of wound. The peptide species can be homolog of trout Ea4-peptide, human Eb-peptide (SEQ ID NO.:1) of pro-IGF-I or a fusion protein comprising the Ea4- or Eb-peptide of pro-IGF-I. It can be administered any wound in a pharmaceutically acceptable composition alone or in combination with other compounds.

The compounds, nucleic acid molecules, polypeptides, and antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

As used herein, the term “wound” is used to refer to tissue and/or cellular damage. The tissue or cellular damage can be caused, for example, by a burn, such as chemical, heat or light; cuts; abrasions; lesions; sores; infection; irritation; inflammation; fibrosis or necrosis.

In one embodiment, the present invention provides a method promoting the proliferation of a fibroblast cell, for example, a human fibroblast, comprising the step of treating a fibroblast with an effective amount of a composition comprising an IGF-I E-domain peptide. In certain embodiments, the peptide species comprises at least one of an rtEa4-peptide, a human Eb-peptide or a combination thereof (e.g., either separately or linked, for example, chemically conjugated or linked contiguously in a single polypeptide chain).

As used herein, the term “E-peptide” (or “E-peptide encoding nucleic acid”) or “E-domain peptide” (or “E-domain peptide encoding nucleic acid”) is used interchangeably to refer to the E-domain of an IGF-1 polypeptide or gene, respectively, of an animal, and portions thereof. In another aspect, the present invention contemplates a fusion protein comprising the amino acid or peptide sequence of an E-domain peptide or homologue of an E-domain peptide of IGF-I, or a protein comprising the E-domain peptide of IGF-I, fused or contiguous with a non-E-domain peptide. For example, in certain embodiments the invention includes fusion proteins comprising a “tag” or indicator portion and an E-peptide portion. In certain aspects the tag or indicator portion can be a peptide adapted for purification purposes, for example, FLAG tag, 6×His tag, or the like. In other aspects, the tag peptide comprises a peptide adapted for providing a signal such as an antibody epitope or a fluorescent peptide. Still other aspects include the fusion of the E-peptide with another peptide that is adapted for mediating subcellular localization or translocation across a cellular membrane, for example, a TAT fusion protein from the HIV virus.

In certain other aspects, the invention includes methods for the treatment of or amelioration of tissue damage and/or disorders related to tissue damage comprising administering an effective amount of the composition of the invention to a subject in need thereof. In certain embodiments, the invention comprises methods for treating tissue damage or wounds, for example, cuts, abrasions, lesions, ulcers, burns, bed sores, gum diseases, mucositis, and the like, comprising administering an effective amount of the therapeutic composition of the invention to a subject in need thereof.

In still other embodiments, the invention comprises therapeutic compositions useful as a surgical adjuvants. In any of the embodiments described herein, the surgical adjuvant composition of the invention can be used or applied as a stand alone therapeutic directly to the surgical site or it can be integrally associated with a surgical or medical implement, for example, the therapeutic of the invention may be conjugated to a polymer-based stent, tube or other implantable device, such that the therapeutic diffuses to the site of action in a controlled manner to accelerate healing and/or to minimize trauma from an invasive surgical procedure. In another embodiment, the therapeutic composition of the invention is applied as, for example, a film or coating to the medical implement such that the therapeutic diffuses into the blood stream or surrounding tissues and/or wears away, and is thereby delivered directly to the site of tissue damage; minimizing or ameliorating the amount of cellular damage that occurs due to the use of the surgical implement.

In still other embodiments, the invention comprises methods for the treatment and/or prevention of deficiencies in tissue repair that occur as a natural side-effect of the aging process (e.g., skin rejuvenation, receding gums, bone degeneration, arthritis, Alzheimers, Parkinsons, and the like). In certain aspects of this embodiment, the invention comprises administering an effective amount of a therapeutic composition of the invention to a subject suffering from age-related deficiencies in tissue repair capacity, tissue integrity, and/or tissue elasticity. In certain embodiments, the age-related deficiency is at least one of wrinkles, crows feet, facial lines, pot marks, scars, fibroids, sun spots, and the like, or combinations thereof.

According to another aspect of the present invention, the peptide species is administered in a pharmaceutical composition comprising the E-peptide species and one or more pharmaceutically acceptable excipients, carriers, and/or adjuvants. In an alternative embodiment, the peptide species is administered to a fibroblast cell by transforming the cells with exogenous nucleic acid that results in expression of an E-peptide in the cell.

In yet another embodiment, the present invention provides a method of promoting the proliferation and migration of a fibroblast comprising the step of administering to the fibroblast a nucleic acid encoding a protein comprising an E-domain peptide of IGF-I. Furthermore, the protein encoded by the nucleic acid administered according to the present invention comprises an a-type E-domain peptide or a b-type E-domain peptide of IGF-I, for example, rtEa4-peptide, hEb-peptide or a combination of both. Alternatively, the nucleic acid encodes a protein that is a homolog of the E-domain peptide of IGF-I, or a fusion protein comprising the E-domain peptide of IGF-I.

In an alternative embodiment, the present invention contemplates a method for promoting the repair of tissue injury in an animal, and/or the repair of cellular injury, comprising administering an effective amount of a composition comprising an E-domain peptide. In certain embodiments, the peptide species comprises at least one of an rtEa4-peptide, a human Eb-peptide or a combination thereof.

Alternatively, the present invention contemplates a method wherein the peptide species comprises a homolog of the E-domain of IGF-I, or a fusion protein comprising the E-domain of IGF-I. In addition, the present invention provides a method wherein the peptide species is administered in a pharmaceutical composition comprising the peptide species and one or more pharmaceutically acceptable excipients, carriers, or adjuvants. In an alternative embodiment, the injury repair occurs by transforming the cells with one or more exogenous nucleic acids that results in expression of an E-domain peptide of IGF-I in the cell. Accordingly, in another embodiment, the invention relates to nucleic acid constructs (i.e., plasmids, vectors, expression constructs, and the like) comprising a nucleic acid encoding for an E-domain polypeptide or fragment thereof, operably linked with a DNA regulatory element, for example, a transcription, and/or replication regulatory element

In yet another embodiment, the present invention contemplates a method for promoting the proliferation and migration of a fibroblast and/or promoting repair of tissue or cellular injury, comprising administering to a cell at least one nucleic acid encoding an E-domain peptide. Preferably, the encoded protein comprises a rtEa4-peptide. Alternatively, the protein comprises an E-domain of human IGF-I. Preferably, the protein comprises an Eb domain of human IGF-I. In another aspect, the protein comprises a homologue of the E-domain of IGF-I, or a fusion protein comprising the E-domain of IGF-I.

In other embodiments, the invention pertains to isolated nucleic acid molecules that encode E-domain peptides, E-domain fusion proteins, and therapeutic compositions comprising the same. The nucleic acids and peptides of the invention can be formed according to any of several well known methods, including, for example, using a nucleic acid or a peptide synthesizer according to standard methods. Alternatively, peptides of the invention can be formed by expressing a nucleic acid construct in a host cell (prokaryotic or eukaryotic) or cell extract, followed by an isolation and purification step.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂, RbCl, liposome, or liposome-protein conjugate can be used. Transformation can also be performed after forming a protoplast of the host cell or by electroporation. These examples are not limiting on the present invention; numerous techniques exist for transfecting host cells that are well known by those of skill in the art and which are contemplated as being within the scope of the present invention.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell. For long-term, high-yield production of recombinant proteins, stable expression is preferred.

Oligonucleotides (e.g.; antisense, GeneBlocs) are synthesized using protocols known in the art as described in Caruthers et al., 1992, Methods in Enzymology 211, 319, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204). The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163).

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above. The use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules and/or other chemical or biological molecules). The treatment of subjects with nucleic acid molecules can also include combinations of different types of nucleic acid molecules.

Recent attention has been focused on the biological activities of the proteolytically-processed polypeptides from post-translational modified peptide hormones. As discussed above, the C-peptide of pro-insulin has long been regarded to be biologically inactive except for a possible role in the folding of the insulin molecule during its post-translational modification. However, Ito et al. (1997) have reported that the C-peptide of pro-insulin was important in restoring vascular and neural dysfunction and Na+/K+-dependent ATPase activity in diabetic rats. Although a synthetic peptide amide of human b-type IGF-I E-peptide has been shown to exert mitogenic activity (Siefried et al., 1992), the biological activity of the native human E-peptides has not previously been identified.

Multiple alternative spliced forms of IGF-I transcript have been identified in mouse and rat (Roberts, et al., Mol. Endocrinol. 1: 243-48, 1987; Shimatsu, et al., J. Biol. Chem. 262: 7894-900, 1987). The alternative splicing of exon 5, resulting in variations in the E-domain of pro-IGF-I (Ea or Eb), has been shown to display developmental regulation and tissue specificity (Lin, et al, J. Endocrinol. 160: 461-67, 1999; Lin et al., Growth Horm IGF Res. 8: 225-33, 1998). Like mature IGF-I, as discussed in general above, the amino acid sequences of mouse or rat E-domains are highly homologous to their human counterparts. The biological significance of this conserved diversity of the E-domain and its differential expression is not clear. However, it is suggestive of potential biological activities associated with E-domain peptides. The presence of glycosylation sites on pro-IGF-I E-domains and the detection of such glycosylated products further suggest potential biological activity of E-domain peptides (Duguay, et al., J. Biol. Chem. 270: 17566-74, 1995). Indeed, a synthetic peptide amide with a 23-amino-acid sequence from the human pro-IGF-Ib E-domain (103-124) has been shown to possess mitogenic activity in human bronchial epithelial cells (Siegfried, et al., Proc. Natl. Acad. Sci. USA 89: 8107-11, 1992).

Thus, the present inventors have demonstrated that novel biological activities are associated with both the rtEa peptides and with the human Eb peptide.

In still other embodiments, the invention comprises a cosmetic composition useful for the repair, regeneration, or restoration of body tissues comprising the therapeutic of the invention and a cosmetically suitable carrier or excipient. In one aspect of this embodiment, the invention encompasses a method of enhancing the appearance of skin comprising administering an effective amount of the therapeutic composition of the invention in a cosmetic to a subject.

The invention further includes a method for screening for a modulator of E-peptide activity. The method includes contacting a test compound with an E-peptide and determining if the test compound binds to said E-peptide. Binding of the test compound to the E-peptide indicates the test compound is a modulator of activity, or of latency or predisposition to the aforementioned disorders or syndromes.

The molecules of the instant invention can be used as pharmaceutical agents. Pharmaceutical agents prevent, inhibit the occurrence, or treat (alleviate a symptom to some extent, preferably all of the symptoms) a disease state in a subject.

In any of the embodiments described herein, the E-domain peptide can be combined with a pharmaceutically acceptable excipients, adjuvant, or carrier, a protein, lipid, glycol, glyceride, antioxidant, saccharide, or the like; another biologically active agent, for example, an analgesic or anti-inflammatory (e.g., aspirin, an NSAID, a COX inhibitor, or the like), an anesthetic, an anti-angiogenic (e.g., angiostatins or endostatin), a chemotherapeutic, a cytotoxic agent (e.g., antimetabolites, antibiotics, alkylating agense, alkaloids), an antineoplastic agent (e.g., cytokines, antibodies, vaccines), a hormonal agent (e.g., LHRH agonists, antiandrogens, anti-estrogens, aromatase inhibitors, progestagens), or the like.

In addition, the E-domain treatment in any of the embodiments described herein may be delivered via any pharmacological acceptable route, for example, oral, topical, anal, intravenous, enteral, parenteral, subcutaneous, intramuscular, transdermal, intracapsular, intraspinal, intracranial, or the like. Furthermore, in any of the embodiments described herein the E-domain peptide may be delivered in any pharmaceutically acceptable forms, for example, a powder, a liquid (e.g., a spray, intravenous solution), a gel, a polymeric matrix, a pill or capsule (e.g., a controlled release capsule, a time release capsule, or both), subdermal implant, and the like.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the complex of the invention.

“Derivatives” are compositions formed from the native compounds either directly, by modification, or by partial substitution.

“Analogs” are nucleic acid sequences or amino acid sequences that have a structure similar to, but not identical to, the native compound.

Derivatives or analogs of the nucleic acids or proteins of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS IN MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present invention, genes are homologous if the nucleic acid sequences are sufficiently similar to allow recombination and/or hybridization under low stringency conditions.

As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA.

Furthermore, one of ordinary skill will recognize that “conservative mutations” also include the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS 1N MOLECULAR BIOLOGY, F. M. Ausubel et al., eds., Current Protocols, a joint venture between Greene Publishing Associates, Inc. and John Wiley & Sons, Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47.

In yet another embodiment, a nucleic acid of the invention is expressed in mammalian cells using a mammalian expression vector. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

Nucleic acid molecules of the invention can also be administered in the form of suppositories, e.g., for rectal administration of the drug or via a catheter directly to the bladder itself. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols. Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid. A pharmacological composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By pharmaceutically acceptable formulation is meant, a composition or formulation that allows for the effective distribution of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly(DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly(ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutical composition of the invention is formulated to be compatible with its intended route of administration. Administration routes which lead to systemic absorption include, without limitations: parenteral, e.g., intravenous, intradermal, subcutaneous, oral (e.g., inhalation), transdermal (i.e., topical), transmucosal, intraperitoneal, inhalation, intrapulmonary, intrathecal, intramuscular, and rectal administration. In addition, there is provided a pharmaceutical formulation comprising a nucleic acid or peptide molecule of the invention and a pharmaceutically acceptable carrier. A molecule of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the therapeutic complex of the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Sustained-release preparations can be prepared. Suitable examples of sustained-release preparations include semipermeable matrices of solid hydrophobic polymers containing an E-peptide, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

A therapeutically effective dose refers to that amount of the therapeutic sufficient to result in amelioration or delay of symptoms. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the negatively charged polymer.

The amount of active ingredient that can be combined with the carrier materials to produce a single dosage form varies depending upon the host treated and the particular mode of administration. Dosage unit forms generally contain between from about 1 mg to about 1000 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The composition can also be administered to a subject in combination with other therapeutic compounds to increase the overall therapeutic effect. The use of multiple compounds to treat an indication can increase the beneficial effects while reducing the presence of side effects. In additional embodiments, the therapeutic of the invention may comprise one or more biologically active ingredients such as, Analgesics, Antacids, Antianxiety Drugs, Antiarrhythmics, Antibacterials, Antibiotics, Anticoagulants and Thrombolytics, Anticonvulsants, Antidepressants, Antidiarrheals, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Cold Cures, Corticosteroids, Cough Suppressants, Cytotoxics, Decongestants, Diuretics, Expectorants, Hormones, Hypoglycemics (Oral), Immunosuppressives, Laxatives, Muscle Relaxants, Sedatives, Sex Hormones, Sleeping Drugs, Tranquilizer, Vitamins or a combination thereof.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; Dropulic et al., 1992, J. Virol., 66, 1432 41; Weerasinghe et al., 1991, J. Virol., 65, 5531 4; Ojwang et al., 1992, Proc. Natl. Acad. Sci. USA, 89, 10802 6; Chen et al., 1992, Nucleic Acids Res., 20, 45819; Sarver et al., 1990 Science, 247, 1222 1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector.

In one aspect the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al., 1993, Methods Enzymol., 217, 47 66; Zhou et al., 1990, Mol. Cell. Biol., 10, 4529 37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 315; Ojwang et al., 1992, Proc. Natl. Acad. Sci. USA, 89, 10802 6; Chen et al, 1992, Nucleic Acids Res., 20, 45819; Yu et al., 1993, Proc. Natl. Acad. Sci. USA, 90, 6340 4; L'Huillier et al., 1992, EMBO J., 11, 4411 8; Lisziewicz et al., 1993, Proc. Natl. Acad. Sci. U.S.A., 90, 8000 4; Thompson et al., 1995, Nucleic Acids Res., 23, 2259; Sullenger & Cech, 1993, Science, 262, 1566).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

The above referenced compositions are given by way of example and are not to be construed as limiting on the scope of the present claims. Indeed, the E-domain therapeutic of the present invention can be delivered in any number of pharmaceutically acceptable forms and routes, which will be readily apparent to those of ordinary skill in the art. The present invention is further illustrated and described by the following examples, which are not intended to limit the scope of the invention in any way.

### Example 1

### Cell Lines and Cell Culture Conditions

The following conditions were used for routine maintenance of cell cultures. Human breast cancer cells (MCF-7, ZR-75-1 and MDA-MB-231 cells) were obtained from American Type Cell Collection (ATCC, Rockville, Md.). They were cultured in F12/DMEM supplemented with 10% fetal bovine serum (FBS) and 10 ng/ml of insulin. Human colon cancer cells (HT-29 cells from ATCC) were cultured in F12/DMEM supplemented with 10% FBS; human HT1080 cells cultured in RPMI 1640 medium with 10% FBS; human hepatoma cells (HepG2 cells from ATCC), transformed human embryonic kidney cells (293GP cells, kindly provided by Dr. J. C. Burns at the University of California-San Diego) and human neuroblastoma cells (SK-N-F1 cells from ATCC) cultured in DME medium with high concentration of glucose and 10% FBS; and Poeceliposis lucida hepatoma cells (HC, kindly provided by Dr. Larry Hightower at the University of Connecticut) cultured in CO.sub.2-independent medium supplemented with 10% FBS. All cell cultures were incubated at 37.degree. C. under a humidified atmosphere of 5% CO.sub.2, except HC cells that were incubated at 30.degree. C. All tissue culture media and supplements used in this study were purchased from Gibco-BRL (Rockville, Md.).

Cells under various treatment conditions were maintained at 37.degree. C. in a 5% CO.sub.2 incubator and observed from 30 minutes to 72 hours after incubation (synthetic human Ea- and Eb-peptide were chemically synthesized at the Biotechnology Center, University of Connecticut). For treatment with synthetic hEa, hEb peptide or hlGF-I, 1.times.10.sup.5 cells were plated in each well of a 12-well culture plate in DMEM/F12 (1:1) supplemented with 0.4-3.2.mu.M of synthetic hEa-, hEb-peptide and/or 5-10 nM of hlGF-I.

### Example 2

### Transfection of Cells with a Construct Comprising Trout Ea-4 cDNA

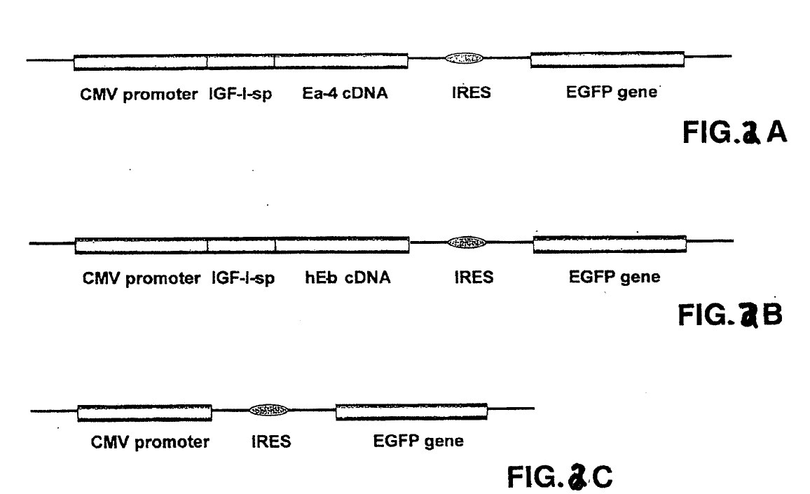

Two gene constructs, CMV-IGF-1-sp-Ea-4-cDNA-IRES-EGFP and CMV-IRES-EGFP were used in the transfection studies. The first construct (FIG. 2A) contained Ea-4 peptide cDNA (with a signal peptide sequence derived from hlGF-I), a ribosome re-entry site (IRES) and an enhanced green fluorescence protein (EGFP) marker gene. The other gene construct (FIG. 9C) contained IRES and EGFP, but did not contain Ea-4-peptide cDNA. The expression of both gene constructs was driven by a promoter from CMV. Vectors containing the recognized control sequences and marker gene are available from commercial sources (CLONTECH, Palo Alto, Calif.).

Transfection of the cells was accomplished as follows. MDA-MB-231 cells were cultured in F12/DMEM supplemented with 10% FBS and 10 ng/ml of insulin to 90% confluence. About 5.times.10.sup.6 cells were harvested and resuspended in 1 mL of serum-free F12/DMEM containing 20.mu.g of un-linearized constructs. The cells were electroporated in a BRL Cell-Porator using the following settings: low •, 1180 micro Faraday (•F) capacitance, and two pulses at 200 volts. Following electroporation, cells were resuspended in 12 mL of fresh growth medium and seeded into a 6-well plate to recover. Permanent transfectants expressing green fluorescence protein (GFP) were enriched in a medium containing neomycin (G418) at 1 mg/mL for ten days and followed with 500·g/mL for continuous maintenance. Individual green cell clones of transfectants were isolated from the enriched population by the method of serial dilution.

The presence of the transgene and the expression of Ea-4 (SEQ ID NO: 2) in transfectants were determined by PCR and comparative RT-PCR assays following conditions described by Greene et al. (1999). Ea-4-peptide specific primers used in the amplification were: forward primer (5′-CTTGTGGCCGTTTACGTC-3′) (SEQ ID NO 6); AND reverse primer (5′-GCACAGCACCCAGACAAG-3′) (SEQ ID NO 7).

Results of PCR analysis of genomic DNA isolated from transfectants confirmed that clones E-9 and E-15 contained Ea-4-peptide cDNA, whereas control EGFP clones did not. Comparative RT-PCR analysis showed that about same levels of mRNA for Ea-4-peptide were detected in both clones E-9 and E-15, while no Ea-4 mRNA was detected in EGFP control clones. Soft Agar Colony Formation assay showed that while EGFP-transfected MDA-MB-231 cell clones formed colonies on soft agar medium, none of the Ea-4-peptide gene transfected MDA-MB-231 clones (i.e., E9 and E15) form any colonies in soft agar medium, suggesting that the colony formation activity of MDA-MB-231 cells is abolished by the Ea-4 peptide. Furthermore, obvious morphological changes were also observed in MDA-MB-231 transfected cells expressing the Ea-4-peptide gene (FIG. 10E).

Because the signal peptide sequence of human IGF-I was also included in the Ea-4 cDNA transgene, the Ea-4 peptide produced by the transfected cell clones would be secreted into the medium. To confirm this, media isolated from EGFP clone and E15 clone were tested for their activities to induce morphological change in untransfected MDA-MB-231 cells. Results presented in FIGS. 10F, 10G and 10H showed that while medium isolated from E15 clone was able to induce the morphological change of untransfected MDA-MB-231 cells, medium isolated from EGFP cells could not. Therefore, these results rule out the possibility that any contaminant from the E. coli extract could result in the anti-tumor activity observed above.

### Example 3

### Transfection of Cells with a Construct Comprising hEb cDNA

Two gene constructs, CMV-IG F-1-sp-hEb-cDNA-IRES-EGFP and CMV-IRES-EGFP were used in the transfection studies. The first construct (FIG. 2B) contained hEb peptide cDNA (with a signal peptide sequence of hlGF-1), a ribosome re-entry site (IRES) and an enhanced green fluorescence protein (EGFP) marker gene. The other gene construct (FIG. 2C) contained IRES and EGFP, but did not contain hEb-peptide cDNA. The expression of both gene constructs was driven by a promoter from CMV.

Transfection of the cells was accomplished as follows. MDA-MB-231 cells were cultured in F12/DMEM supplemented with 10% FBS and 10 ng/ml of insulin to 90% confluence. About 5×10⁶ cells were harvested and resuspended in 1 mL of serum-free F12/DMEM containing 20 μg of un-linearized constructs. The cells were electroporated in a BRL Cell-Porator using the following settings: low •, 1180 micro Faraday (μF) capacitance, and two pulses at 200 volts. Following electroporation, cells were resuspended in 12 mL of fresh growth medium and seeded into a 6-well plate to recover. Permanent transfectants expressing green fluorescence protein (GFP) were enriched in a medium containing neomycin (G418) at 1 mg/mL for ten days and followed with 500 μg/mL for continuous maintenance. Individual green cell clones of transfectants were isolated from the enriched population by the method of serial dilution.

The presence of the transgene and the expression of hEb in transfectants were determined by PCR and comparative RT-PCR assays following conditions described by Greene et al. (1999). hEb-peptide specific primers used in the amplification were: forward primer (5′-CTTGTGGCCGTTTACGTC-3′) (SEQ ID NO 6); AND reverse primer (5′-GCACAGCACCCAGACAAG-3′) (SEQ ID NO: 7).

### Example 4

### Morphological Changes Induced by Rainbow Trout Ea-4 Peptides and Synthetic Human Analogues

Approximately 1-2×10⁵ of MCF-7, ZR-75-1, HT-29, HepG2, 293GP, HC or SK-N-F1 cells re-suspended in their respective basal medium without fetal bovine serum (FBS) were plated in a 6-well culture chamber. Prior to plating cells, an acid-washed coverslip was placed in each well of the culture chamber. Recombinant rainbow trout E-peptides (rtEa-2, rtEa-3 or rtEa-4 peptide at 0.8 μM), human IGF-I (hIGF-1, 2.5 nM) or the same amount of control protein was added to each well and the cell cultures were incubated at 37° C. under a humidified atmosphere of 5% CO₂. The control protein was prepared from E. coli cells carrying the expression plasmid but without the E-peptide gene according to the purification method described by Tian et al. (1999). Coverslips were removed from the culture chamber 24 hours after initiation of the treatment and observed under an Olympic inverted microscope equipped with differential interference phase contrast objective lenses or phase contrast objective lens (final magnification, 200×). The morphological change assay was performed at least 10 times with different batches of Ea-4 peptide preparations. The concentration of these synthetic peptides tested was 0.4 μM.

### Example 5

### Effect of Inhibition of mRNA and Protein Synthesis on Morphological Changes

To study the effects of alpha-aminitin and cycloheximide, known inhibitors of RNA and protein synthesis, respectively, on morphological changes induced by rtEa-4-peptide, about 1-2×10⁵ of ZR-75-1 and 293GP cells, re-suspended in their respective basal medium without FBS, were plated in a 6-well culture chamber. Prior to plating the cells, an acid-washed coverslip was placed in each well of the culture chamber. Each culture was treated with recombinant trout Ea-4-peptide at 0.8 μM, and with either alpha-aminitin at 10 μg/mL (an RNA synthesis inhibitor) or with cycloheximide at 1.0 μg/mL (a protein synthesis inhibitor). The cell cultures were incubated at 37° C. under a humidified atmosphere of 5% CO₂. Coverslips were removed from the culture chamber 24 hours after initiation of the treatment and observed under an Olympic inverted microscope equipped with differential interference phase contrast objective lenses (final magnification, 200×). The viability of the inhibitor-treated cells was further determined by a dye extrusion assay.

To determine whether the morphological changes induced by the Ea-4 peptides requires synthesis of new proteins or of RNA, 293GP and MCF-7 cells were cultured under the same conditions as described above. Ea-4 peptide-induced morphological changes in 293GP and MCF-7 cells were abolished by treatment with cycloheximide or alpha-aminitin. These results suggest that the morphological change induced by the Ea-4 peptide might result from expression of genes that were activated and/or inactivated during oncogenic transformation or tumor development. This conclusion is further substantiated by results of studies on microarray screening of a collection of human EST's that indicate that the Ea-4 peptide up- and/or down-regulated the expression of a series genes related to cell attachment, proliferation and invasion of MDA-MB-231 cells.

### Example 6

### Relative Activity of Trout Ea-2, Ea-3, and Ea-4 Peptides

In examining the biological activity of E-peptides of human pro-IGF-1, the present inventors have determined that hEb peptide (SEQ ID NO:1), like rainbow trout Ea-4 SEQ ID NO:2) peptide, evidences novel and unique activities, apart from the know functions of mature IGF-1. The in vitro effective concentration of synthetic hEb peptide (0.4-3.2·M) is within a similar range as that of the recombinant rtEa-4 peptide.

Cells were again cultured as described above. Twenty-four hours after treatment with 0.8 μM of E-peptides or the control protein, the cells were observed under an Olympic inverted microscope equipped with differential interference phase contrast objective lenses (200× magnification). Although both Ea-2- and Ea-4-peptides were able to induce morphological change in 293GP or ZR-75-1 cells, the Ea-3 peptide failed to induce any visible morphological change under the identical culture conditions. This observation indicates that the domain of the E-peptide responsible for the induction of morphological change in the 293GP or ZR-75-1 is not present in the Ea-3-peptide. To confirm this hypothesis, synthetic peptides specific to Ea-1-, Ea-2-, Ea-3- and Ea-4-peptide (Ea-1sp, Ea-2sp, Ea-3sp and Ea-4-sp) specific sequence (see FIG. 1) were prepared and tested for their activities to induce morphological change in ZR-75-1 cells. Cells were cultured in F12/DME medium without FBS as described above. Twenty-four hours after treatment with 0.8 μM of synthetic peptide containing Ea-1 sp, Ea-2sp, Ea-3sp or Ea-4-sp specific sequence or the control protein, the cells were observed under an Olympic inverted microscope equipped with differential interference phase contrast objective lenses (200× magnification). Ea-2sp and Ea-4-sp, but not Ea-1sp and Ea-3sp at 0.4 μM were able to induce a morphological change. These results indicate that the active domain of the Ea-peptide resides within the 12 amino acid residues of Ea-2-peptide.

### Example 7

### Morphological Changes and Inhibition Assays Using Human Eb Peptide

Neuroblastoma SK-N-F1 cells (10⁵) were seeded into 12-well culture plates in DMEM/F12 (1:1) supplemented with 0-3.2 μM of synthetic hEb-peptide, or buffer control, and incubated at 37° C. in a 5% CO₂ humidified incubator.

For inhibition studies, cells were pre-incubated with vehicle (0.1% DMSO), or 10-50 μM of the MEK inhibitor PD98059 (Promega, Madison, Wis.), or 10 nM-1 μM of the PI-3K inhibitor wortmannin (Sigma), or 10-50 μM of LY294002 (Promega, Madison, Wis.), for one hour prior to the addition of 3.2 μM hEb-peptide.

Cell images were taken by random sampling at various time points using a MicroMAX CCD camera (Princeton Instruments, Bozeman, Mont.). Approximately 1000 cells were analyzed from each treatment, carried out in triplicate. Cells with neurites longer than one cell body diameter (>20 μm for SK-N-F1 cells) were scored as positive neurite-bearing (Fagerstrom, et al., Cell Growth Differ., 7: 775-85, 1996; Morrione, et al., Cancer Res. 60: 2263-72, 2000). The percentage of neurite-bearing cells and the respective length of neurites were measured with reference to a stage micrometer and analyzed using the public domain NIH Image program (developed at the U.S. National Institutes of Health and available on the Internet at http://rsb.info.nih.gov/nih-image).

### Example 8

### Morphological Differentiation (Neurite-Like Growth) in Neuroblastoma Cells

SK-N-F1 neuroblastoma cells are characterized as poorly differentiated embryonal cells with an epithelial-like morphology. In examining the biological activities of synthetic hEa and hEb peptides of pro-IGF-I, the present inventors determined that hEb peptides induce morphological changes in SK-N-F1 cells, whereas synthetic hEa-peptide, like mature IGF-I, lacks this activity. Cells treated with the mature hlGF-I (5 nM) remained rounded and formed aggregated clusters, similar to the morphology of the control cells, and that of the cells treated with 3.2 μM synthetic hEa-peptide alone, or in combination with 5 nM hlGF-I. In contrast, SK-N-F1 cells treated with synthetic hEb-peptide (1.6 μM or 3.2 μM) differentiated into a neuron-like morphology with one or multiple neurite-like processes and a relatively small cell body. The treatment of mature hlGF-I and hEb-peptide combined further enhanced the formation of neurite-like processes, and resulted in a 20-30% increase in the percentage of neurite-bearing cells at 1-4 hours after stimulation (quantitative data not shown). The activities of hEb-peptide are identical to those of rainbow trout Ea-4-peptide described above.

To further characterize the dose-response relationship and time course of hEb peptide in inducing neurite-like process outgrowth, SK-N-F1 neuroblastoma cells were treated with various amounts (0 to 3.2 μM) of hEb peptide over a course of 72 hours. Images of cells were taken by random sampling after 1 h, 6 h, 24 h, 48 h and 72 h of incubation. The average length of neurite-like process outgrowth was measured by random sampling of more than 1000 cells at various time points. The neurite-like processes started to be visible as early as 0.5-1 hour after the addition of hEb peptide. The effect of hEb peptide in inducing neurite-like outgrowth was dose-dependent, as evident from a comparison of cells treated with 0.4 μM hEb peptide with those treated with 0.8-3.2 μM hEb peptide over 24-72 hours. The maximum effect of hEb peptide was observed 48 hours after the addition of 3.2 μM hEb peptide, with an average neurite length of 50 to 60 μm (Table 1, below).

TABLE 1
DOSE RESPONSE AND TIME COURSE STUDIES
ON hEb-PEPTIDE INDUCED NEURITE GROWTH

| [hEb-pep- | | | | | |
|---|---|---|---|---|---|
| tide] | Neurite length (μm)^{‡} | | | | |
| (μM) | 2 h | 6 h | 24 h | 48 h | 72 h |
| 0 | 19 ± 0.1 | 21 ± 4.6 | 24 ± 2.2 | 24 ± 0.3 | 24 ± 1.3 |
| 0.4 | 30 ± 1.6 | 31 ± 7.6 | 29 ± 5.2 | 35 ± 0.9 | 32 ± 3.1 |
| 0.8 | 33 ± 2.1 | 36 ± 0.6 | 38 ± 3.9 | 43 ± 1.0 | 41 ± 2.5 |
| 1.6 | 33 ± 3.8 | 38 ± 3.5 | 39 ± 1.8 | 47 ± 3.9 | 48 ± 2.8 |
| 3.2 | 33 ± 2.8 | 39 ± 2.2 | 45 ± 1.7 | 52 ± 3.7 | 48 ± 2.7 |

^{‡}neurite length shown as mean ± standard deviation determined from more than 1000 cells sampled in triplicate at each time point; a dose-response relationship was observed when comparing cells treated with 0.4 μ.M hEb-peptide and those treated with 0.8-3.2 μM hEb-peptide with statistical differences (P • 0.05) from 24-72 hours; the maximum effect of hEb-peptide was obseved at 48 h after initiation of treatment.

In examining the biological activity of E-peptides of human pro-IGF-I, the present inventors have determined that hEb peptide, like rainbow trout Ea-4 peptide, evidences novel and unique activities, apart from the known functions of mature IGF-I. The in vitro effective concentration of synthetic hEb peptide (0.4-3.2.mu.M) is within a similar range as that of the recombinant rtEa-4-peptide.

### Example 9

### Effects of Trout Ea-4 Peptide on Colony Formation

An obvious change in the characteristics of normal cells after oncogenic transformation is the loss of contact inhibition and anchorage-dependent cell division behavior (Kosaki et al., 1999). This behavioral change in oncogenic transformed or established cancer cells can be easily demonstrated in vitro by a colony formation assay in a semi-solid medium (Dickson et al., 1986).

Colony formation assays were conducted following the method described by Yang (1975). About 2×10⁴ of HT-29 (colon cancer cells) or MDA-MB-231 cells (aggressive breast cancer cells) at log phase were plated in their respective basal medium containing 1.25% FBS and 0.5% purified agar (Difco laboratories), and supplemented with various concentrations (0.4 to 1.6 μM) of the recombinant rainbow trout Ea-4 peptide, or the same amount of the control protein, in 6-well culture chambers. After the medium is solidified, each well is overlaid with 1 mL of the basal medium (containing 1.25% FBS) supplemented with same concentration of the trout Ea-4 peptide. The plates were incubated at 37° C. in a humidified incubator with 5% CO₂ and examined daily under an inverted microscope for 2-3 weeks. Colonies were observed under an Olympic inverted microscope equipped with phase contrast objective lenses (final magnification: 40×). Colonies with sizes •50 μm were scored. The viability of cells at the conclusion of the experiment was confirmed by dye extrusion assay with tryptan blue. The assay was conducted two times.

To confirm whether treatment of transformed cells with the Ea-4 peptide could result in increased attachment of the cells to the culture dish, 293GP cells were cultured in a serum-free basal medium supplemented with Ea-4-peptide (0.8 μM) or 10% FBS, respectively, in 6-well culture chambers. After four days, the culture medium was removed, and cells were rinsed twice with PBS containing 0.02% EDTA, fresh PBS was added, and the culture plates shaken 20 times manually. At the end of shaking, cells cultured in serum-free medium or medium supplemented with FBS detached completely from the culture chamber, while cells cultured in the serum-free medium supplemented with Ea-4 peptide remained attached to the culture chamber. These results clearly showed that rtEa-4 peptide enhances the attachment of oncogenic transformed cells to the culture chamber, similar to the behavior exhibited by untransformed (normal) cells.

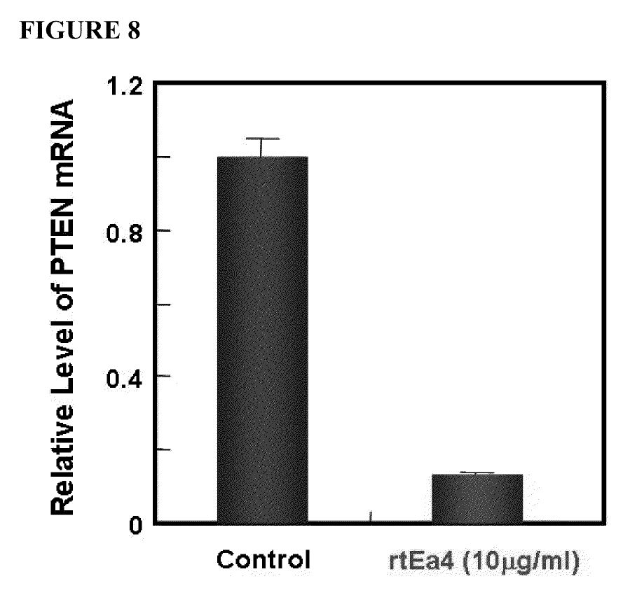

It has been suggested that the malignant growth property of human neuroblastoma cells can be associated with their differentiation status (Martin, et al., J. Pediatr. Surg. 3: 161-64, 1968). Spontaneous resolution has in fact been observed as a result of neuronal differentiation of neuroblastoma cells in vivo (Pahiman, et al., Eur. J. Cancer., 31A: 453-58, 1995). As shown in FIGS. 8A and 8B, many visible colonies were developed from both cancer cell lines grown in the soft agar medium supplemented with 1.25% FBS and the control protein, but fewer colonies were developed from both cell lines cultured in the same medium supplemented with increasing concentrations of recombinant Ea-4 peptide. These results showed that Ea-4 peptide is able to reduce or abolish the anchorage-independent cell division behavior of tumor cells.

### Example 10

### Effects of hEb Peptide on Colony Formation

Poor differentiation and anchorage-independent cell growth are among the hallmarks of poor prognosis in neuroblastoma disease. As discussed above, neuroblastoma cells present a unique system in which the relationship between differentiation and tumorigenesis might be successfully dissected. Loss of proper differentiation is a common theme in cellular transformation in many different types of cancer. Thus, inducing cellular differentiation and intervening growth factor signaling have now been discussed as novel alternative approaches to cancer treatment (Garattini and Terao, Curr. Opin. Pharma., 1: 358-63, 2001; Favoni, de Cupis, Pharmcol. Rev., 52: 179-206, 2000). According to the present invention, hEb peptide, like rainbow trout Ea-4 peptide, induces morphological differentiation in neuroblastoma cells.

The effect of hEb-peptide on in vitro colony formation was tested in the presence or absence of either the mature hlGF-I or fetal bovine serum. Human neuroblastoma SK-N-F1 cells (10⁴) were mixed with 0.4% soft agar supplemented with or without hEb peptide (3.2 μM), IGF-I (5 nM) and/or fetal bovine serum (2.5%), as indicated. The cell mixtures were seeded on top of a solidified basal medium DMEM/F12 (1:1) containing 0.5% agar. Medium supplemented with various peptides or serum were overlaid on top of the solidified cell layer followed by a two-week period incubation at 37° C. in a 5% CO₂ humidified incubator. The percentage of cells formed into colonies with a diameter greater than 100 μm Macpherson, Tissue culture methods and applications, pp 276-80: N.Y. Academic Press, 1973) were scored in triplicate and subjected to Student t-test analysis. At least three independent assays under each treatment conditions were carried out.

Mature hlGF-I (5 nM), like fetal bovine serum (FBS, 2.5%) strongly stimulated colony formation in neuroblastoma cells (SK-N-F1). On the other hand, hEb-peptide (3.2 μM) significantly reduced the percentage of cells grown into colonies with a diameter greater than 100 μm. In the absence of serum and growth factors, inhibition of colony formation by hEb peptide was 64%, similar to that in the presence of hlGF-I (5 nM) (59% inhibition). Colony formation in the presence of FBS (2.5%) was inhibited by 73%. According to the present invention, in a manner similar to that demonstrated for rtEa-4 peptide, the hEb peptide of human pro-IGF-I exhibits an inhibitory effect on anchorage-independent growth by 59-73%. This activity is in sharp contrast to the stimulatory effect of mature IGF-I.

To further confirm the effect of hEb-peptide on reduction or elimination of malignant growth of cancer cells, aggressive breast cancer cells, MDA-MB-231 and ZR-75-1 cells, were transfected with a hEb-peptide gene construct. hEb gene transfected cells, namely hEb A, hEb B, hEb C and hEb H, exhibited a morphology similar to that of untransfected MDA-MB-231 treated with synthetic hEb-peptide. Furthermore, results of the colony formation assay showed that, like treatment of cancer cells with synthetic hEb-peptide, the colony formation activities of hEb gene transfected cells were greatly reduced or diminished completely.

Normally, adherent cells require anchorage to extracellular matrix (ECM) to survive and proliferate. This anchorage dependency is primarily mediated by integrins that are responsible for engaging cell-ECM interaction and thus activating the growth- and survival-promoting signals. Tumor cells, including neuroblastoma cells, are generally resistant to apoptosis induced by loss of attachment to ECM and cannot only survive but grow independently of anchorage. According to the present invention, the hEb peptide of human pro-IGF-I restores the anchorage dependency for cell survival and cell division in neuroblastoma cells. These results suggest, without limiting the present invention, that hEb-peptide induced signaling may act collaboratively and converge with extracellular adhesion signaling pathways in regulating cell survival and division. The results provided herein also indicate that the hEb peptide, but not the hEa peptide of human pro-IGF-I induces morphological differentiation and inhibits anchorage-independent growth in human neuroblastoma cells. A similar nature and range of biological activities have been shown with Ea-4 peptide of rainbow trout pro-IGF-I. Thus, E-peptides of pro-IGF-I are not only biologically active but are functionally conserved in fish and humans. Furthermore, the data disclosed herein also indicate, without limiting the scope of the present invention, that these conserved E-peptide activities might be mediated by conserved signal transduction mechanisms.

### Example 11

### Promotion of Fibroblast Proliferation and Tissue Repair or Wound Healing

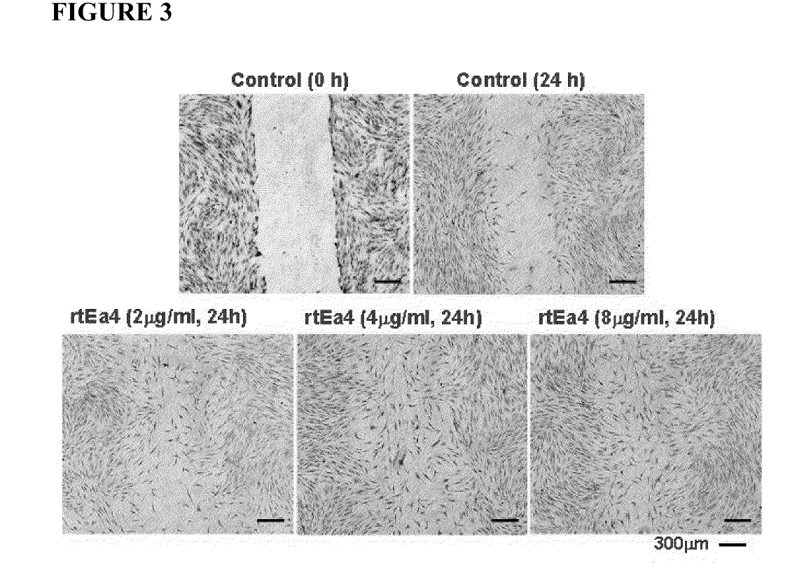

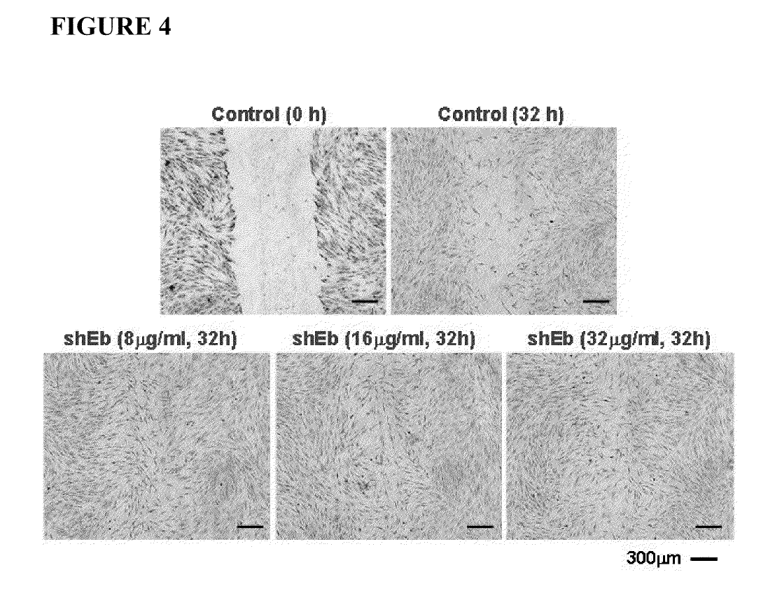

FIGS. 3 and 4 show that trout Ea4- or human Eb-peptide of pro-IGF-I enhances the scratch wound of human fibroblast, CCD-1112SK, cells (ATCC® Number: CRL-2429). For a detailed review of CCD-1112SK culture and etiology, see, Hovatta O, et al. A culture system using human foreskin fibroblasts as feeder cells allows production of human embryonic stem cells. Hum. Reprod. 18: 1404-1408, 2003; Ellerstrom C, et al. Facilitated expansion of human embryonic stem cells by single-cell enzymatic dissociation. Stem Cells 25: 1690-1696, 2007, the teachings of which are incorporated herein by reference in their entirety.

Human CCD-1112SK cells were grown to monolayer in growth medium in 6-well plates. Scratch wounds were created by using the end of an one-ml pipette. Following the wounds were created, various amounts of trout Ea4-peptide (2-8 μg/ml) or human Eb-peptide (8-32 μg/ml) were added. After incubation for 24 h or 32 h, medium was removed, cells were stained with Giemsa stain and observed under a microscope. The results showed that rtEa4- or hEb-peptide exhibited a dose-dependent promotion of wound in CCD-112SK cells

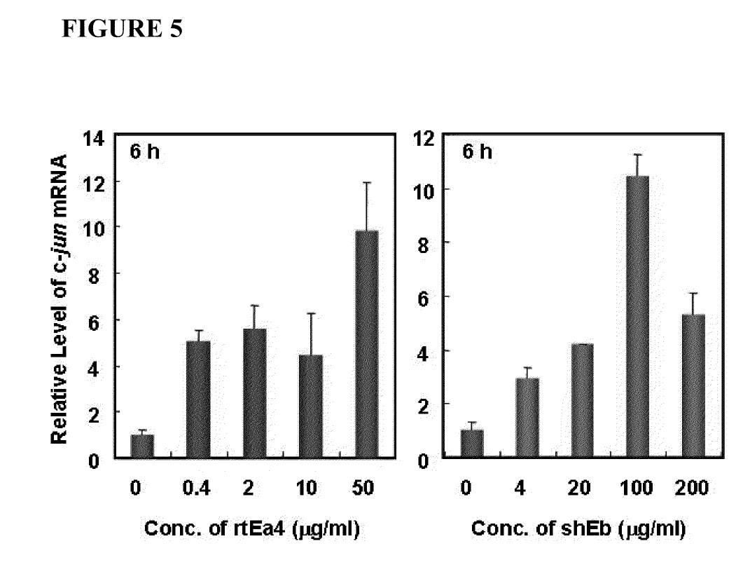

FIG. 5 shows the Induction of c-jun mRNA levels by trout Ea4- or human Eb-peptide in CCD-1112SK cells. It has been shown that cells under wound closure exhibited high levels of c-jun mRNA. If trout Ea4- or human Eb-peptide can promote the closure of scratch wound of CCD-1112SK cells, it should induce c-jun mRNA in CCD-1112SK cells. Human fibroblast cells (CCD-1112SK) were grown to monolayer in 6-well plates, scratches were made with an one-ml pipette tip, and various amounts of E-peptides were supplemented into the growth medium. After 6 hours, the cells were harvested and total RNA extracted by the guanidinium thiocyanate-phenol-chloroform method. Levels of c-jun and β-actin mRNAs were determined by real-time quantitative PCR(RTQ-PCR) method and data expressed as: Relative mRNA level=2^{-[S·CT-C·CT]}. Each data point was the average of at least 6 determinations. The levels of c-jun mRNA in the scratched wound of CCD-1112SK cells were significantly induced by E-peptides. These results further support the discovery that trout Ea4- or human Eb-peptide of pro-IGF-I promotes wound closure

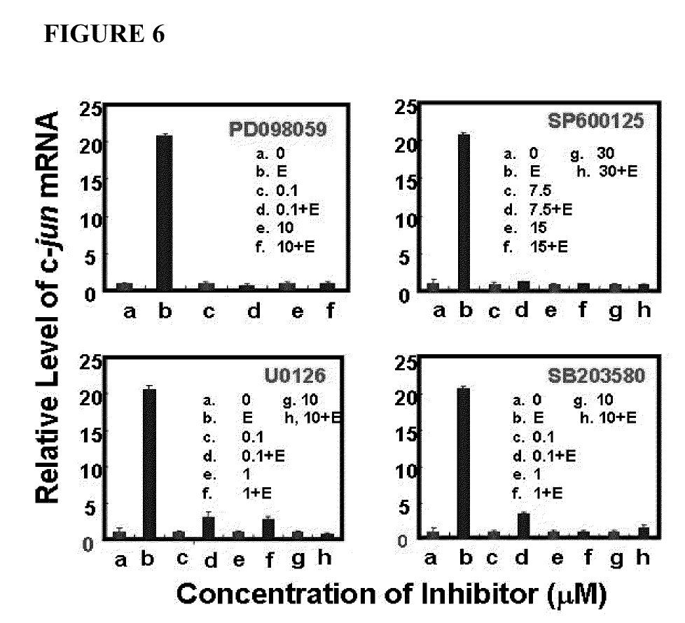

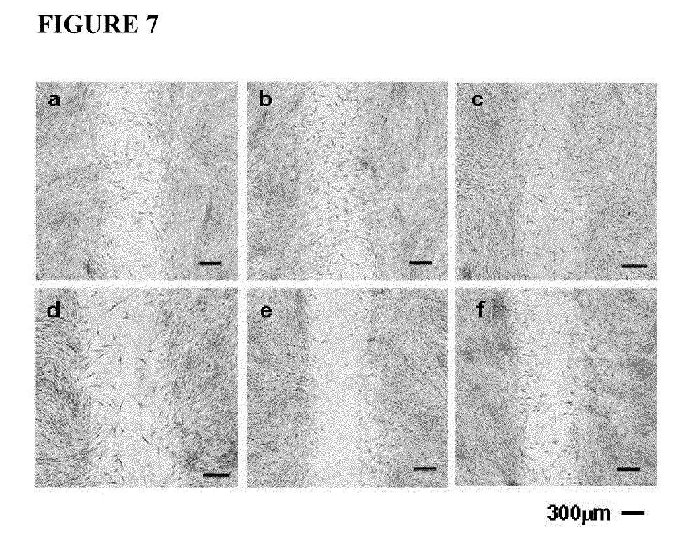

FIGS. 6 and 7 show that troutEa4- or human Eb-peptide induced wound closure is inhibited by inhibitors of signal transduction pathways. Results of experiments on wound healing showed that the process of wound healing is regulated via signal transduction pathways. If E-peptide, indeed, can promote wound closure in CCD1112SK cells, its wound closure activity should be sensitive to inhibitors of Mek1/2 (UO126 and PD09805), JNK (SP600125) and P38 MAPK (SB203580).

Human fibroblast cells (CCD-112SK) were grown to monolayer in 6-well plates, scratches were made with an one-ml pipette tip, treated with various concentrations of signal transduction pathway inhibitors for 1 hour and followed by 6 hours with rtEa4-peptide (10 μg/ml). Cells were harvested, and total RNA extracted with the guanidinium-thiocyanate-chloroform method. Levels of c-jun and b-actin mRNAs were determined by quantitative real-time RT-PCR assay. Relative mRNA level=2^{-[S·CT-C·CT]}. Each data point was the average of at least 6 determinations. Parallel samples were incubated for 24 hours and stained with Giemsa stain for observation under a microscope. The data showed that the rtEa4-peptide induced c-jun mRNA level and wound closure were suppressed by inhibitors of signal transduction pathways. These results further substantiate wound closure effort exerted by trout Ea4- or human Eb-peptide.

FIG. 6 shows the inhibition of PTNE mRNA in CCD-1112SK cells by trout Ea4- or human Eb-peptide. It has been reported that the level of PTEN mRNA is greatly reduced in fibroblast cells in the course of wound closure. If by trout Ea4- or human Eb-peptide is able to promote scratch wound closure in CCD-1112SK cells, this peptide should also suppress the level of PTEN mRNA. Human fibroblast cells (CCD-1112SK) were grown to monolayer in 6-well plates, scratches were made with an one-ml pipette tip, and treated with 10 μg/ml of rtEa-peptide. After 6 hours incubation, cells were harvested and total RNA extracted by the guanidinium thiocyanate-phenol-chloroform method. Levels of PTEN and β-actin mRNAs were determined by RTQ-PCR assay. Relative mRNA level=2^{-[S·CT-C·CT]}. Each data point was the average of at least 6 determinations. The level of PTEN mRNA in the scratched wound of CCD-1112SK cells was significantly suppressed by rtEa4-peptide. These results further support the discovery that trout Ea4- or human Eb-peptide promotes the closure of scratch wound of CCD-1112SK cells.

While this invention has been particularly shown and described with references to exemplary and preferred embodiments thereof, it will be understood by those skilled in the art that various changes in form and details may be made therein without departing from the scope of the present invention encompassed by the appended claims.

1. A method for the promotion of wound repair comprising administering to a wound site an effective amount of a composition comprising an Insulin-like Growth Factor-1 (IGF-1) E-domain peptide (E-peptide) together with at least one of a pharmaceutically acceptable carrier, excipient or adjuvant, wherein the composition promotes repair of the wound.

2. The method of claim 1, wherein the composition comprises at least one E-domain peptide having the amino acid sequence as set forth in SEQ ID NO:2, having the amino acid sequence as set forth in SEQ ID NO:1, or a combination of both.

3. The method of claim 2, wherein the composition comprises a fusion protein having an E-domain peptide joined in a contiguous polypeptide chain with a non-E-domain peptide.

4. The method of claim 1, wherein the wound is at least one of a burn, cut, abrasion, lesion, sore, infection, irritation, inflammation, fibrosis or necrosis.

5. The method of claim 1, wherein the composition is in a pharmaceutical form adapted for topical administration.

6. The method of claim 5, wherein the pharmaceutical form is at least one member selected from the group consisting of gel, lotion, emulsion, liquid, nebulized spray, foam, and combination thereof.

7. A method for promoting fibroblast proliferation comprising administering a composition comprising an effective amount of an E-domain peptide selected from the group consisting of an Ea4-domain peptide having the amino acid sequence of SEQ ID NO:2, an hEb domain having the amino acid sequence of SEQ ID NO: 1, and a combination thereof, together with at least one of a pharmaceutically acceptable carrier, excipient or adjuvant.

8. The method of claim 3, wherein the non-E-domain peptide comprises a fluorescent protein.

9. The method of claim 3, wherein the non-E-domain peptide comprises a TAT polypeptide or portion thereof.

10. The method of claim 1, wherein the tissue damage is due to a surgical procedure or device.

11. The methods of claim 1, wherein the composition comprises a cosmetically suitable carrier.

12. A surgical adjuvant for the amelioration of tissue damage due to a surgical procedure, surgical device or surgical implant comprising an effective amount of an E-domain peptide together with at least one of a pharmaceutically acceptable carrier, excipient or adjuvant.
